(19) United States
(12) Patent Application Publication
(10) Pub. No.: US 2019/0374117 A1
(43) Pub. Date: Dec. 12, 2019

(54) DETECTION DEVICE FOR ATRIAL FIBRILLATION AND OPERATING METHOD THEREOF

(71) Applicant: PixArt Imaging Inc., Hsin-Chu County (TW)

(72) Inventor: Yuan-Hsin LIAO, Hsin-Chu County (TW)

(21) Appl. No.: 16/003,849

(22) Filed: Jun. 8, 2018

(51) Int. Cl. A61B 5/024; A61B 5/00; A61B 5/0255

(52) U.S. Cl. CPC A61B 5/02405; A61B 5/02433; A61B 5/725; A61B 5/7267; A61B 2562/04; A61B 5/7282; A61B 5/7246; A61B 5/7275; A61B 5/0255

(57) ABSTRACT

There is provided an operating method of an AF detection device including a reference model construction step and a continuous detection step. In the reference model construction step, heartbeat waveforms of a PPG signal of a user are classified to construct a personal reference model. In the continuous detection step, current heartbeat waveforms of a PPG signal of the same user is compared with the personal reference model to identify whether each of the heartbeat waveforms is an AF waveform.

### BACKGROUND

### 1. Field of the Disclosure

This disclosure generally relates to a continuous detection for atrial fibrillation (AF) and, more particularly, to a fast response detection device capable of identifying whether each heartbeat waveform in a photoplethysmography (PPG) signal is an AF waveform and an operating method thereof.

### 2. Description of the Related Art

Presently, the atrial fibrillation (AF) is identified using Electrocardiogram (ECG). However, electrodes are required in the detection of ECG, and thus continuous long-term detection is not suitable due to the inconvenience of carrying the electrodes all day long. Accordingly, the intermittent measurement (e.g., each time for several minutes) is generally conducted by using electrodes to detect the ECG such that it is difficult to record the circadian rhythm of a user.

The best way to perform a long-term monitoring is to use a wearable electronic device. However, the aforementioned electrodes for measuring ECG are generally difficult to be integrated with the wearable electronic device.

It is known that a photoplethysmography (PPG) signal is detectable using a wearable electronic device. However, signals obtained through the wearable electronic device generally contain noises caused by the relative movement between a detection device and the skin surface such that the identification accuracy is degraded. Meanwhile, different users generally have different AF signals such that the implementation of continuous and high accurate detection is not easy.

Accordingly, it is necessary to provide a fast respond detection device and an operating method thereof that are adaptable to a portable electronic device or a wearable electronic device and can personalize the AF waveform for increasing the identification accuracy.

### SUMMARY

The present disclosure provides a atrial fibrillation (AF) detection device and an operating method thereof capable of personalizing AF waveforms corresponding to different users to improve the identification accuracy.

The present disclosure further provides a fast respond atrial fibrillation (AF) detection device and an operating method thereof capable of performing the AF identification on each heartbeat waveform of a PPG signal.

The present disclosure provides an atrial fibrillation (AF) detection device configured to construct a personal AF model. The AF detection device includes a light sensor, a processor and a memory. The light sensor is configured to detect light from a skin surface and output a photoplethysmography (PPG) signal. The processor is coupled to the light sensor, and includes a filter and a model constructor. The filter is configured to retrieve a predetermined interval of PPG signal having an AF feature in the PPG signal. The model constructor is configured to perform a wave segmentation and a wave classification on the predetermined interval of PPG signal, and construct a personal reference model according to classified AF waveforms. The memory is configured to store the personal reference model.

The present disclosure further provides an atrial fibrillation (AF) detection device including a memory, a light sensor and a processor. The memory is configured to previously record a personal reference model of a user. The light sensor is configured to detect light from a skin surface to output a photoplethysmography (PPG) signal. The processor is coupled to the light sensor, and configured to segment the PPG signal to a plurality of heartbeat waveforms, and compare the plurality of heartbeat waveforms with the personal reference model to identify whether each of the plurality of heartbeat waveforms is an AF waveform.

The present disclosure further provides an operating method of an atrial fibrillation (AF) detection device. The operating device includes the steps of: constructing a reference model according to a photoplethysmography (PPG) signal, and identifying AF waveforms in a PPG signal. The constructing step includes the steps of: retrieving a predetermined interval of PPG signal having an AF feature in the PPG signal; performing a wave segmentation and a wave classification on the predetermined interval of PPG signal; and constructing a personal reference model according to classified AF waveforms. The identifying step includes the steps of: segmenting the PPG signal to a plurality of heartbeat waveforms; comparing the plurality of heartbeat waveforms with the personal reference model; and identifying whether each of the plurality of heartbeat waveforms is an AF waveform.

The AF detection device of the present disclosure is suitable to the continuous long-term detection on a portable electronic device or a wearable device, and the detection result is informed to a user through an indication unit.

### BRIEF DESCRIPTION OF THE DRAWINGS

Other objects, advantages, and novel features of the present disclosure will become more apparent from the following detailed description when taken in conjunction with the accompanying drawings.

FIG. 1 is a block diagram of an AF detection device according to one embodiment of the present disclosure.

FIG. 2 is a flow chart of constructing a reference model in an operating method of an AF detection device according to one embodiment of the present disclosure.

FIGS. 3A-3F are schematic diagrams of an operating method of an AF detection device according to one embodiment of the present disclosure.

FIG. 4 is a schematic diagram of one heartbeat waveform.

FIG. 5 is a flow chart of performing a continuous detection in an operating method of an AF detection device according to one embodiment of the present disclosure.

FIGS. 6A and 6B are schematic diagrams of comparing real-time heartbeat waveforms with a personal reference model by an AF detection device according to one embodiment of the present disclosure.

FIG. 7 is a block diagram of an AF detection device according to another embodiment of the present disclosure.

### DETAILED DESCRIPTION OF THE EMBODIMENT

It should be noted that, wherever possible, the same reference numbers will be used throughout the drawings to refer to the same or like parts.

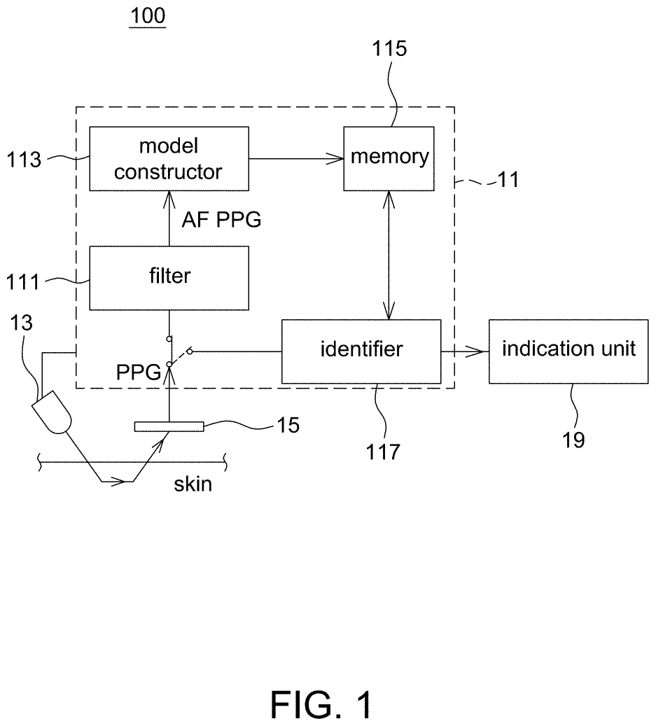

Referring to FIG. 1, it is a block diagram of an atrial fibrillation (AF) detection device 100 according to one embodiment of the present disclosure. The AF detection device 100 includes a processor 11, a light source 13, a light sensor 15 and an indication unit 19. In one non-limiting embodiment, the light sensor 13 is not included in the AF detection device 100 of the present disclosure, and light for illuminating the skin surface is provided by a light source arranged in other devices or provided by ambient light.

In one non-limiting embodiment, the processor 11, the light source 13 and the light sensor 15 are encapsulated in the same package to form a detection chip, which is integrated in a portable device or a wearable device. The detection chip detects a skin surface via a surface of the device. The portable device and the wearable device includes an indication unit 19 used to represent the appearance, a number of accumulated times and a temporal distribution of AF waveforms using video or sound. For example, the indication unit 19 is a display used to show a number or a time variation of the appearance of AF waveforms; or, the indication unit 19 is a speaker used to play an alert sound indicating the appearance or a number of accumulated times of AF waveforms, but not limited thereto. The indication unit 19 is a proper device as long as it is able to inform the user regarding the AF waveforms.

The AF detection device 100 is used to previously construct a personal AF reference model, and is actuated by executing a software and/or hardware. For example, the AF detection device 100 is triggered by pressing a button on or selecting an APP or icon shown a display of a portable device or a wearable device. The AF detection device 100 is further used to identify the atrial fibrillation according to a photoplethysmography signal (sometimes referred to PPG signal below) of the user. In one non-limiting embodiment, when the AF detection device 100 starts to detect the PPG signal, the continuous detection for the atrial fibrillation is performed automatically.

The light source 13 is a coherent light source, a partial coherent light source or a non-coherent light source, e.g., a light emitting diode or a laser diode. The light source 13 emits light suitable to be partially absorbed by skin tissues to illuminate a skin surface, e.g., emitting red light, green light and/or infrared light. After the light emitted by the light source 13 passes through skin tissues, light which is partially absorbed by the skin tissues is emergent from the skin surface to be detected by the light sensor 15.

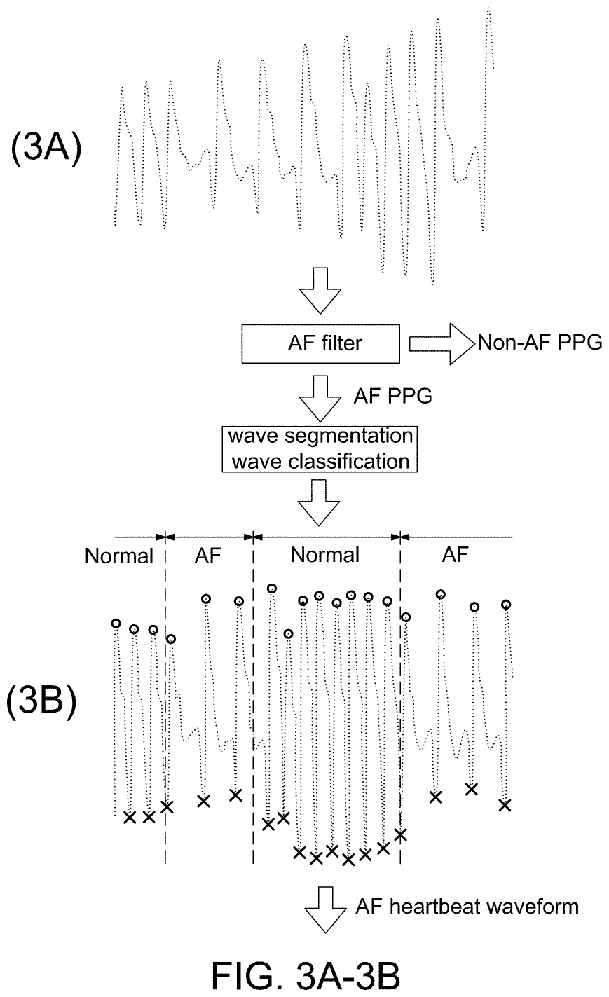

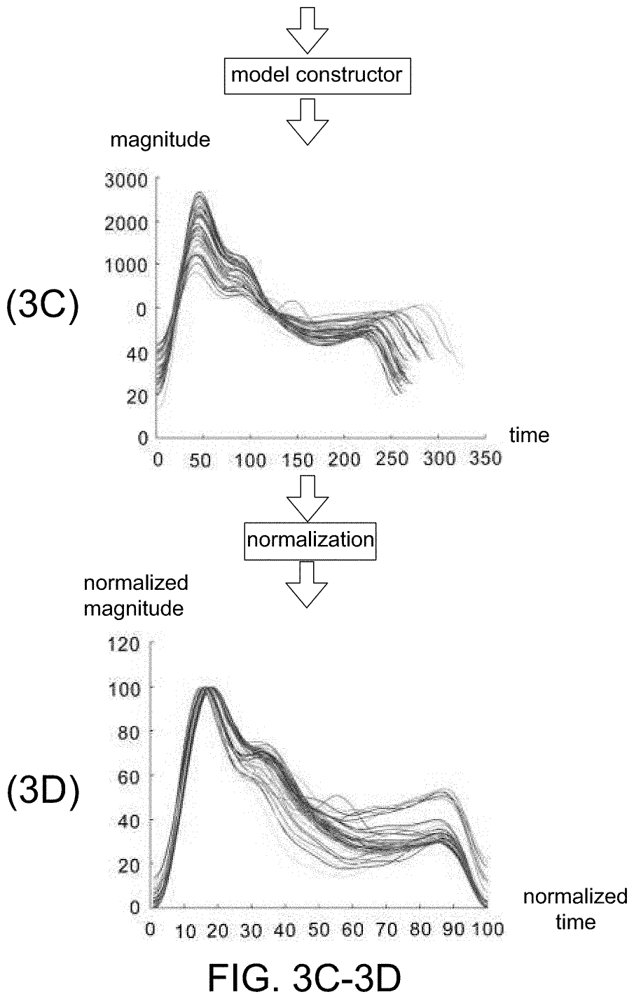

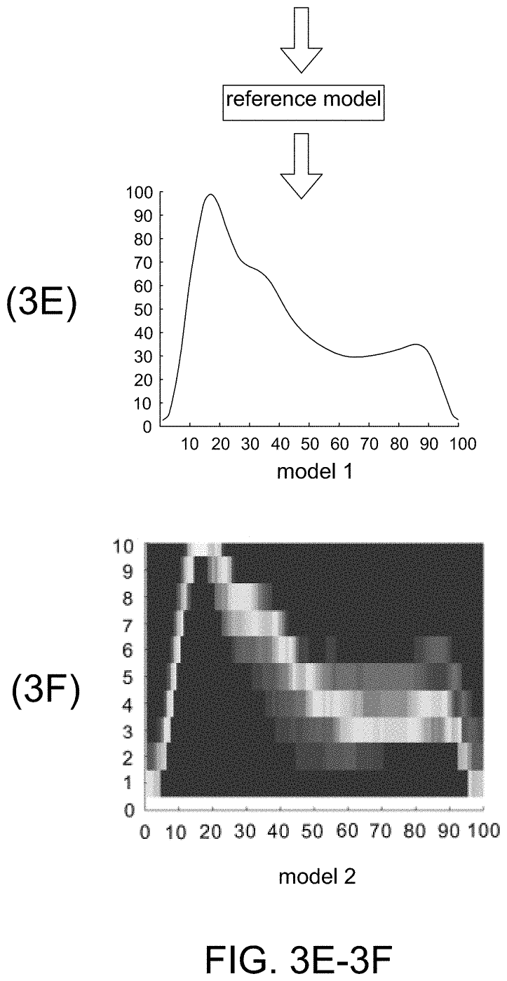

The light sensor 15 includes, for example, a single photodiode or a photodiode array. When the light sensor 15 includes a photodiode array, the light sensor 15 is a CMOS image sensor or a CCD image sensor having multiple photodiodes arranged in a matrix. The light sensor 15 is used to detect light emergent from the skin surface to output a PPG signal. When the light sensor 15 includes a single photodiode, said single photodiode outputs one PPG signal (e.g., shown in FIG. 3A). When the light sensor 15 includes a photodiode array, multiple PPG signals may be outputted (e.g., each photodiode outputting one PPG signal) or an average of the multiple PPG signals is outputted (e.g., averaged by the circuit or by a digital signal processor, DSP).

The processor 22 is, for example, a microcontroller unit (MCU), a central processing unit (CPU) or an application specific integrated circuit (ASIC) that performs the operation thereof using software and/or hardware. The processor 11 includes a filter 111, a model constructor 113, a memory 115 and an identifier 117, wherein the memory 115 includes a volatile memory and/or a nonvolatile memory used to record the algorithm and operating parameters temporarily or permanently, e.g., recording a personal reference model of a user and thresholds (illustrated by an example below).

In the present disclosure, during constructing the reference model, a personal reference model is constructed using the filter 111 and the model constructor 113 to be stored in the memory 115; and during the continuous detection, AF waveforms are identified using the identifier 117 and the memory 115. For example, the processor 11 further includes a switching device or a multiplexer used to send, under different modes, the PPG signal detected by the light sensor 15 to the filter 111 (in the model construction mode) or the identifier 117 (in the continuous detection mode). In addition, it is appreciated that although FIG. 1 shows different operations being executed by different functional blocks, it is only intended to illustrate but not to limit the present disclosure. Operations executed by every functional block FIG. 1 are considered being executed by the processor 11.

For example, a portable device or a wearable device has a button or a display on which an icon is shown for triggering an APP to enter the model construction mode, and said model construction mode is performed before the AF detection device 100 performs the fast response identification.

In the process of constructing the reference model, the filter 111 is used to retrieve a predetermined interval of PPG signal having an AF feature within the PPG signal acquired by the light sensor 15, wherein said predetermined interval of PPG signal is a PPG signal section within 3 to 5 minutes and having the AF feature. More specifically, when the filter 111 identifies that the PPG signal does not contain any AF feature, the PPG signal is not sent to the model constructor 113. Only when AF features are identified in the PPG signal, the filter 111 sends a predetermined interval (e.g., 3 to 5 minutes which is determined according to an occurring frequency of the atrial fibrillation) of PPG signal to the model constructor 113 for constructing a personal reference model. It is appreciated that not every heartbeat waveform within the predetermined interval has the AF feature. A PPG signal section within a predetermined interval during which a number of waveforms having the AF feature is higher than a predetermined number is selected to be sent to the model constructor 113.

The filter 111 identifies the AF feature using, for example, a normalized root mean square of successive RR difference (nRMSSD) technique or a Shannon entropy technique, e.g., referred to, but not limited to, following references

“A Novel Application for the Detection of an Irregular Pulse using an iPhone 4S in Patients with Atrial Fibrillation,” McManus et al., 2013.

“Smart detection of atrial fibrillation,” Krivoshei et al., 2017.

“Identification of Atrial Fibrillation by Quantitative Analyses of Fingertip Photoplethysmogram,” Tang et al., 2017.

Next, after receiving the predetermined interval of PPG signal, the model constructor 113 performs a wave segmentation and a wave classification on the predetermined interval of PPG signal, and constructs a personal reference model according to classified AF waveforms.

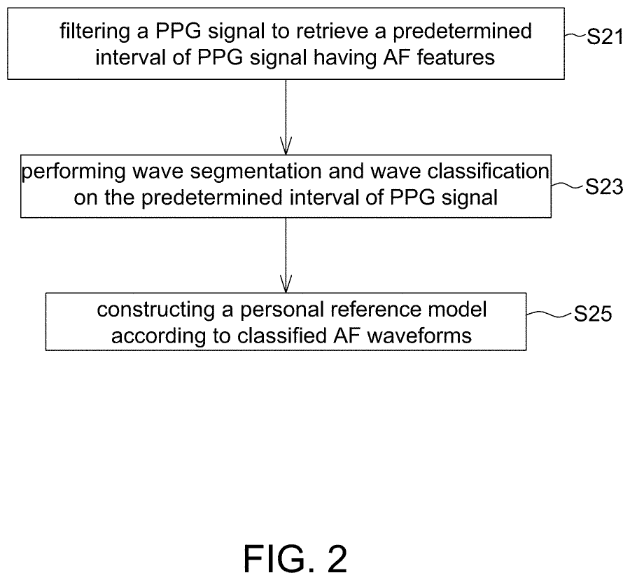

For example referring to FIG. 2, it is a flow chart of constructing a reference model in an operating method of an AF detection device according to one embodiment of the present disclosure, including the steps of: retrieving a predetermined interval of PPG signal having an AF feature in the PPG signal (Step S21); performing a wave segmentation and a wave classification on the predetermined interval of PPG signal (Step S23); and constructing a personal reference model according to classified AF waveforms (Step S25).

Referring to FIGS. 3A-3F, they are schematic diagrams of constructing a personal reference model according to an embodiment of the present disclosure.

Firstly, the processor 11 controls the light source 13 to turn on and turn off corresponding to the detection of the light sensor 15. In the case that the light source 13 is not integrated in the AF detection device 100, the light source 13 illuminates light continuously. The light sensor 15 outputs a PPG signal, e.g., as shown in FIG. 3(A), using a predetermined detection frequency to the filter 111. In this mode, the PPG signal is not sent to the identifier 117.

Step S21: The filter 111 filters the PPG signal, e.g., using the aforementioned nRMSSD method or Shannon entropy method, to retrieve a predetermined interval (e.g., 3 to 5 minutes) of PPG signal having an AF feature (e.g., shown as AF PPG in FIG. 3A-3B) among the PPG signal from the light sensor 15. Data of the PPG signal without the AF feature (e.g., shown as Non-AF PPG in FIG. 3A-3B) is not used to construct the reference model.

Step S23: Next, the model constructor 113 performs segmentation and classification on the predetermined interval of PPG signal. For example, FIG. 3B shows normal waveforms (indicated as normal) and AF waveforms (indicated as AF) after the classification. The wave segmentation is performed according to systolic peaks or diastolic peaks of the predetermined interval of PPG signal. For example, waveform between two successive systolic peaks or two successive diastolic peaks in the PPG signal is considered as a heartbeat waveform, e.g., waveform between two successive diastolic peaks being taken as the heartbeat waveform herein.

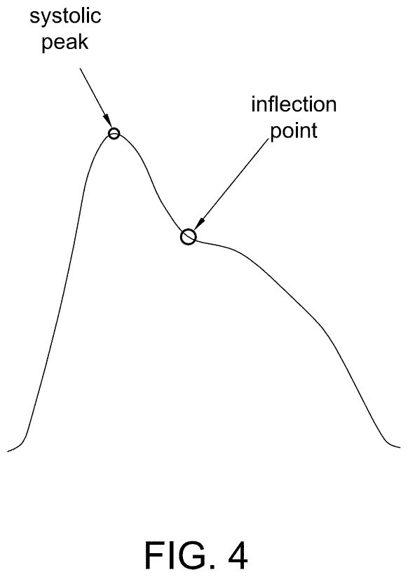

The wave classification is performed according to at least one of a systolic peak and an inflection point of segmented heartbeat waveforms from the predetermined interval of PPG signal. For example FIG. 4 shows one heartbeat waveform which has one systolic peak and one inflection point. In one non-limiting embodiment, when the systolic peak of one heartbeat waveform is not clear, e.g., referring to unfit for diagnosis in “Optimal Signal Quality Index for Photoplethysmogram Signals” by Mohamed Elgendi, 2016, and/or one heartbeat waveform has more than 2 (including 2) inflection points, said one heartbeat waveform is defined as an AF waveform herein; on the contrary, said one heartbeat waveform is defined as a normal waveform. In another embodiment, Skewness is another parameter used to distinguish AF and non-AF waveforms, e.g., also referring to the document “Optimal Signal Quality Index for Photoplethysmogram Signals”. It is clear from FIG. 3B) that the predetermined interval of PPG signal contains AF waveform section and non-AF waveform section.

Step S25: Next, the model constructor 113 constructs a personal reference model according to multiple AF waveforms, including continuous and non-continuous waveforms. In one non-limiting embodiment, the model constructor 113 overlaps the multiple AF waveforms, e.g., the AF waveforms indicated in FIG. 3(B), as shown in FIG. 3(C). Because every heartbeat waveform generally has different RR intervals, to increase the accuracy, the overlapped data shown in FIG. 3(C) is preferably normalized as shown in FIG. 3(D), and thus normalized magnitudes are obtained with a same normalized time. The model constructor 113 constructs a personal reference model according to the normalized data, e.g., FIG. 3(D).

In one non-limiting embodiment, the model constructor 113 calculates an average of normalized data at each normalized time point in FIG. 3(D) to generate an average waveform of multiple classified AF waveforms, shown as model 1 in FIG. 3E).

In one non-limiting embodiment, the model constructor 113 converts FIG. 3(D) to a probability map, shown as model 2 in FIG. 3(F), in which the region having a lighter color indicates higher probability, and the region having a darker color indicates lower probability. When a real-time heartbeat waveform is overlapped with the probability map, the probability of every data point is obtained.

In the present disclosure, the above average waveform and/or probability map is used to represent a personal reference model of a user and stored in the memory 115. After the personal reference model is stored in the memory 115, the reference model construction mode is ended.

Next, an operating method of the detection process is illustrated below. In the detection process, the memory 115 has already stored a personal reference model as shown in FIG. 3(E) or 3(F). The processor 11 is also used to control the light source 13 to turn on and off corresponding to the detection of the light sensor 15. The light sensor 15 outputs a photoplethysmography signal (sometimes referred to PPG signal below) at a sampling frequency. Herein, the PPG signal is sent to the identifier 117 which is used to segment the PPG signal to multiple continuous heartbeat waveforms. The identifier 117 then compares the segmented multiple heartbeat waveforms with the personal reference model to identify whether each of the segmented multiple heartbeat waveforms is an AF waveform.

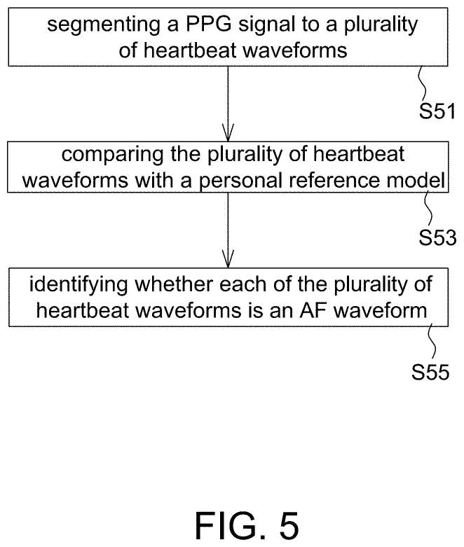

Referring to FIG. 5, it is a flow chart of performing a continuous detection in an operating method of an AF detection device according to one embodiment of the present disclosure, including the steps of: segmenting a PPG signal to a plurality of heartbeat waveforms (Step S51); comparing the plurality of heartbeat waveforms with a personal reference model (Step S53); and identifying whether each of the plurality of heartbeat waveforms is an AF waveform or not (Step S55).

Step S51: Firstly, the identifier 117 segments the PPG signal. Similar to Step S23, the identifier 117 performs the wave segmentation according to systolic peaks or diastolic peaks of the PPG signal, and because the classifying method have been described above, details thereof are not repeated herein. It is appreciated that the segmentation in Step S51 is identical to that in Step S23.

Step S53: The identifier 117 then compares every segmented real-time heartbeat waveform with the stored personal reference model, e.g., shown in FIG. 3(E) or 3(F), depending on the model being stored. Similarly, as RR intervals of every real-time heartbeat waveform have some differences, preferably the identifier 117 also normalizes the real-time heartbeat waveform at first similar to FIGS. 3(C) and 3(D), and then the comparison is conducted. It should be mentioned that the heartbeat waveform is illustrated by real-time heartbeat waveform here is for distinguishing from those used in the reference model construction step.

When the personal reference model is an average waveform as FIG. 3(E), the identifier 117 calculates similarity or correlation of every real-time heartbeat waveform with the average waveform, wherein the similarity is calculated using techniques such as mean square error (MSE), absolute error, dynamic time warping or other conventional methods without particular limitations.

If the personal reference model is a probability map as FIG. 3(F), the identifier 117 calculates a probability value of each of the plurality of real-time heartbeat waveforms according to the probability map. It is assumed that one real-time heartbeat waveform contains multiple amplitude data a1, a2, . . . at. The identifier 117 calculates the probability value using Equation (1):

Probability Value=ln P(a1)+ln P(a2)+ . . . +ln P(at) Equation (1)

Equation (1) is a summation of a natural logarithm of probability of each amplitude data P(a1), P(a2), P(at) which is determined according to a position of corresponding amplitude data a1, a2, . . . at in the probability map.

Step S55: The memory 115 has already stored with the similarity threshold or probability threshold. The identifier 177 compares the calculated result (i.e., probability value) of each real-time heartbeat waveform with the similarity threshold or probability threshold (depending on the personal reference model being used). When the calculated result of one real-time heartbeat waveform exceeds (larger than or smaller than depending on the calculation method being used) the threshold, it means that said one real-time heartbeat waveform is identified as an AF waveform. The identifier 177 then informs the indication unit 19 to represent the appearance or accumulated times of the AF waveforms.

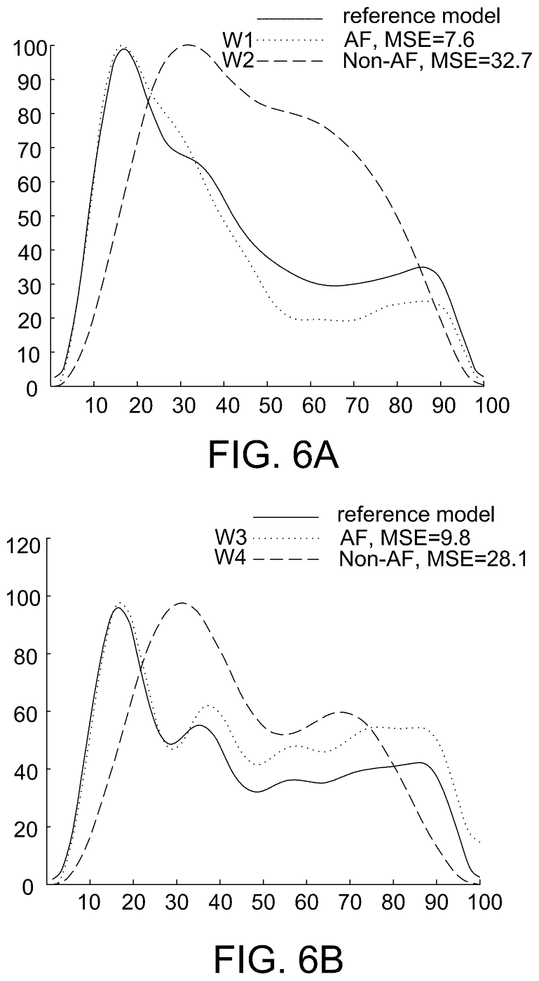

For example, FIG. 6A is a schematic diagram of two waveforms W1 and W2 of a first user as well as an average waveform. It is assumed the mean square error (MSE) is used to represent the similarity herein. The waveform W1 has an MSE=7.6 with respect to the average waveform (i.e., the personal reference model), and the waveform W2 has an MSE=32.7 with respect to the average waveform. If a similarity threshold is selected between MSE=10-20 (but not limited to), an AF waveform is confirmed when the MSE is smaller than the similarity threshold, and a heartbeat waveform is not an AF waveform when the MSE is larger than the similarity threshold. FIG. 6B is a schematic diagram of two waveforms W3 and W4 of a second user as well as an average waveform. In other words, when one heartbeat waveform has a higher similarity with the average waveform which is used as a personal reference mode, said one heartbeat waveform is more likely identified as an AF waveform.

In the present disclosure, the indication unit 19 is arranged in a way that each time an AF waveform appears, and a hint is provided, e.g., showing by the display or a sound being played. In addition, the indication unit 19 is further arranged to represent an accumulated number of times or a number variation of the appearance of AF waveforms within a predetermined time interval. In addition, results represented by the indication unit 19 is further recorded in the memory 115 for being read later by the user. For example, the electronic device adopting the AF detection device of the present disclosure has a wireless communication function such that the record stored in the memory 115 can be read by an external computer for analyzing and post-processing.

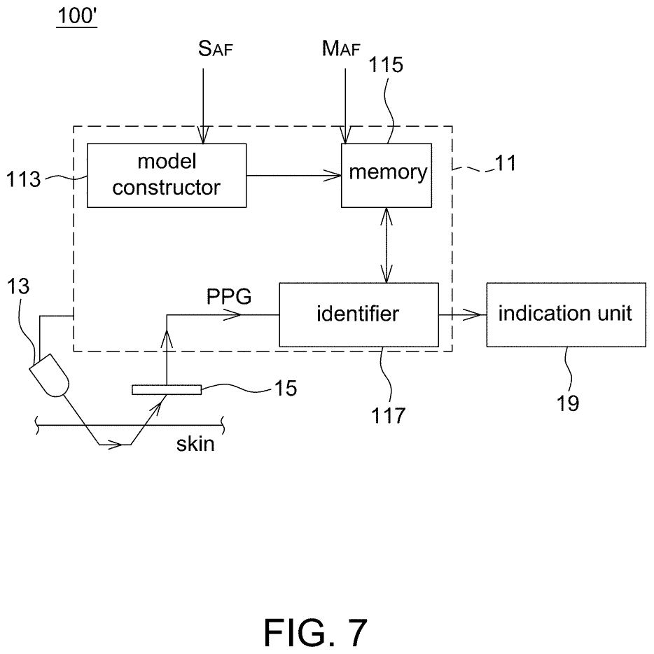

Referring to FIG. 7, it is a block diagram of an AF detection device 100′ according to another embodiment of the present disclosure, wherein identical components in FIGS. 1 and 7 are indicated by identical numerical references. The difference between the AF detection device 100′ and the AF detection device 100 in FIG. 1 is that the AF detection device 100′ in FIG. 7 does not have a filter. The model constructor 113 constructs a personal reference model using the method mentioned above according to an external waveform signal S_{AF}, which is a PPG signal or ECG signal, within a predetermined interval. That is, the source signal for constructing the personal reference model is not acquired by the light sensor 15 of the AF detection device 100′. In another embodiment, the AF detection device 100′ does not include the model constructor 113, and the personal reference model M_{AF} is constructed by an external computer system and directly stored in (e.g., via wireless communication or internet) the memory 115, e.g., the external computer system using the constructing method of the present disclosure mentioned above. The identifier 117 compares the PPG signal with a pre-stored average waveform or probability map to identify an AF waveform, wherein the operation of the identifier 117 has been described above and thus details thereof are not repeated herein.

It should be mentioned that although a reflective type (i.e., the light source and the light sensor being arranged at a same side of the skin) detecting device is used as an example herein, the present disclosure is not limited thereto. In other embodiments, the AF detection device implements the AF detection using a transmissive type (i.e., the light source and the light sensor being arranged at two different sides of the skin) detecting device.

In addition, to denoise detected PPG signal and improve the detection accuracy, the AF detection device of the present disclosure further adopts other denoising technology. For example, the AF detection device works in conjunction with an accelerometer. After the PPG signal is denoised by using a detection result of the accelerometer, the denoised PPG signal is used in the reference model construction step and the continuous detection step. For example, the AF detection device of the present disclosure includes a green light generator and at least one of a red light generator and infrared light generator. The AF detection device denoises the red light PPG signal and the infrared light PPG signal using a PPG signal detected when the green light generator emits light (referred to green light PPG signal). And then the denoised PPG signal is used in the reference model construction step and the continuous detection step. In addition, it is also possible to adopt the denoising technique using a differential between bright and dark images in the AF detection device of the present disclosure.

It should be mentioned that although the AF detection device of the above embodiments is described by adapting for a single user, but the present disclosure is not limited thereto. The AF detection device of the present disclosure is also adaptable to detect the fast response atrial fibrillation of different users as long as the memory 15 is previously recorded with reference models of multiple users.

As mentioned above, the conventional ECG detection device is not suitable for measuring for a whole day. Accordingly, the present disclosure further provides an AF detection device (e.g., FIGS. 1 and 7) and an operating method thereof (e.g. FIGS. 2 and 5) that previously construct a personal reference model using a reference model construction step, and compare current heartbeat waveforms in the PPG signal of a user with the personal reference model to identify whether each of the current heartbeat waveforms of the user is a AF waveform or not. The AF detection device of the present disclosure further informs an appearance, a number of accumulated times or a temporal distribution of AF waveforms via an indication unit.

Although the disclosure has been explained in relation to its preferred embodiment, it is not used to limit the disclosure. It is to be understood that many other possible modifications and variations can be made by those skilled in the art without departing from the spirit and scope of the disclosure as hereinafter claimed.

What is claimed is:

1. An atrial fibrillation (AF) detection device, configured to construct a personal AF model, the AF detection device comprising:
a light sensor configured to detect light from a skin surface and output a photoplethysmography (PPG) signal;
a processor coupled to the light sensor, and comprising:
a filter configured to retrieve a predetermined interval of PPG signal having an AF feature in the PPG signal; and
a model constructor configured to perform a wave segmentation and a wave classification on the predetermined interval of PPG signal, and construct a personal reference model according to classified AF waveforms; and
a memory configured to store the personal reference model.

2. The AF detection device as claimed in claim 1, wherein the light sensor comprises a single photodiode or a photodiode array.

3. The AF detection device as claimed in claim 1, wherein the predetermined interval of PPG signal is a PPG signal section within 3 to 5 minutes having the AF feature.

4. The AF detection device as claimed in claim 1, wherein the filter is configured to identify the AF feature using a normalized root mean square of successive RR difference or a Shannon entropy.

5. The AF detection device as claimed in claim 1, wherein the personal reference model is an average waveform or a probability map of a plurality of classified AF waveforms.

6. The AF detection device as claimed in claim 1, wherein the model constructor is configured to perform the wave segmentation according to diastolic peaks of the predetermined interval of PPG signal.

7. The AF detection device as claimed in claim 6, wherein the model constructor is configured to perform the wave classification on segmented heartbeat waveforms according to at least one of a systolic peak and an inflection point of the segmented heartbeat waveforms in the predetermined interval of PPG signal.

8. An atrial fibrillation (AF) detection device, comprising:
a memory configured to previously record a personal reference model of a user;
a light sensor configured to detect light from a skin surface to output a photoplethysmography (PPG) signal; and
a processor coupled to the light sensor, and configured to segment the PPG signal to a plurality of heartbeat waveforms, and compare the plurality of heartbeat waveforms with the personal reference model to identify whether each of the plurality of heartbeat waveforms is an AF waveform.

9. The AF detection device as claimed in claim 8, wherein the light sensor comprises a single photodiode or a photodiode array.

10. The AF detection device as claimed in claim 8, wherein the personal reference model is an average waveform or a probability map of a plurality of classified AF waveforms.

11. The AF detection device as claimed in claim 10, wherein the processor is configured to calculate similarity or correlation between each of the plurality of heartbeat waveforms and the average waveform.

12. The AF detection device as claimed in claim 10, wherein
the processor is configured to calculate a probability value of each of the plurality of heartbeat waveforms according to the probability map, and
the plurality of heartbeat waveforms are continuous heartbeat waveforms.

13. The AF detection device as claimed in claim 8, further comprising:
an indication unit configured to represent an appearance, a number of accumulated times or a temporal distribution of the AF waveforms, and
a light source configured to illuminate the skin surface.

14. The AF detection device as claimed in claim 8, further comprising a model constructor configured to previously construct the personal reference model according to a predetermined interval of PPG signal, which is outputted by the light sensor, having an AF feature.

15. An operating method of an atrial fibrillation (AF) detection device, the operating method comprising:
constructing a reference model according to a photoplethysmography (PPG) signal, the constructing comprising:
retrieving a predetermined interval of PPG signal having an AF feature in the PPG signal;
performing a wave segmentation and a wave classification on the predetermined interval of PPG signal; and
constructing a personal reference model according to classified AF waveforms; and
identifying AF waveforms in a PPG signal, the identifying comprising:
segmenting the PPG signal to a plurality of heartbeat waveforms;
comparing the plurality of heartbeat waveforms with the personal reference model; and
identifying whether each of the plurality of heartbeat waveforms is an AF waveform.

16. The operating method as claimed in claim 15, wherein the wave segmentation is performed according to diastolic peaks of the predetermined interval of PPG signal.

17. The operating method as claimed in claim 16, wherein the wave classification is performed on segmented heartbeat waveforms according to at least one of a systolic peak and an inflection point of the segmented heartbeat waveforms in the predetermined interval of PPG signal.

18. The operating method as claimed in claim 15, wherein the personal reference model is an average waveform or a probability map of a plurality of classified AF waveforms.

19. The operating method as claimed in claim 18, wherein the comparing comprises:
calculating similarity or correlation between each of the plurality of heartbeat waveforms and the average waveform.

20. The operating method as claimed in claim 18, wherein the comparing comprises:
calculating a probability value of each of the plurality of heartbeat waveforms according to the probability map.
